(19) United States
(12) Patent Application Publication
(10) Pub. No.: US 2022/0065474 A1
(43) Pub. Date: Mar. 3, 2022

(54) ENVIRONMENT MANAGEMENT SYSTEM

(71) Applicant: Crosby & Belle Yoga, LLC, Pittsburgh, PA (US)

(72) Inventor: Tyler Paytas, Pittsburgh, PA (US)

(73) Assignee: Crosby & Belle Yoga, LLC, Pittsburgh, PA (US)

(21) Appl. No.: 17/458,227

(22) Filed: Aug. 26, 2021

Related U.S. Application Data
(60) Provisional application No. 63/071,861, filed on Aug. 28, 2020.

(51) Int. Cl. F24F 8/22; B01D 46/00; B01D 46/42; B01D 46/46; B01D 46/44; A61L 9/20

(52) U.S. Cl. CPC F24F 8/22; A61L 2209/14; B01D 46/0032; B01D 46/4263; B01D 46/46; B01D 46/442; B01D 46/448; B01D 46/444; B01D 46/446; A61L 9/20; B01D 2279/40; B01D 2279/50; A61L 2209/12; A61L 2209/111; B01D 46/0028

(57) ABSTRACT

The present disclosure is directed to systems, apparatuses, and methods to monitor and modulate air quality parameters, such as temperature, humidity, oxygen, and carbon dioxide in an interior area.

### BACKGROUND

### Technical Field

The present disclosure relates to the monitoring and control of various air quality and environmental parameters in an interior area.

### Description of the Related Art

Air contaminants may be present in room air as particle phase pollutants such as dust particles and airborne bacteria and mold. These particulates may range in size from 1/100 microns to a few hundred microns. Air contaminants may also be present in room as gaseous phase pollutants, such as Volatile Organic Compounds (VOCs) which may be as small as a few angstroms to the nano-scale.

To deal with air contaminants different techniques for purification have been used. Filtration is one of the methods used to purify gases such as room air. High efficiency particulate air (HEPA) filters have been employed to remove particulate contaminants from the air. Another method used to remove contaminants form the air includes generating high voltage by electrostatic precipitator or by emitting negative ions, which further charges up the suspended particulates and dust matters in the air, and collects the negatively charged suspended particulates and dust matters by the neutral or positively charged surfaces may also be employed.

Purified air is of increased importance during times of cardiovascular and aerobic strain when the body inhales more air and benefits from greater delivery of oxygen to the body.

### BRIEF SUMMARY

The present disclosure is directed to a comprehensive interior environment management unit that includes a plurality of integrated systems that monitor air quality, temperature, number of individuals, etc. and adjusts aspects of the management unit to maintain a consistent air quality in the space. The interior environment may be any interior environment, but is specifically designed for an interior exercise environment shared by multiple individuals, such as in a class setting. While the interior environment management system may be implemented in a variety of exercise environments, like spin or stationary bike classes, and dance classes, implementing this system in heated yoga classes can improve the experience of each participant and help manage the health and longevity of the space.

The system is designed to accommodate a series of classes in a day, such that there is a cycle that the system manages over a 24 hour period. During open hours, there may be 3-8 cycle of the system associated with a series of 1-1.5 hour classes. The system will monitor the environment during the classes and make appropriate class environment adjustments, like maintaining a selected temperature and air quality. The system will also have an after hours or reset cycle that is performed when there are no participants or other staff within the environment, such as UV treatments to eliminate or manage certain bacteria, mold, and viruses.

The environment management system is implemented in an enclosed space that will include at least one doorway or entryway for participants. In many circumstances, the enclosed space will also include at least one window or opening that can be manually or automatically opened to allow fresh air into the enclosed space from an exterior environment, which is preferably outdoor air, i.e. natural ventilation.

In a heated yoga environment, a heater or heating unit is included that is coupled to an air intake that brings in air from outside of the enclosed space. An environment treatment unit is coupled between the air intake and the heating unit to process the new air. The system is configured to maintain positive pressure in the space during a class. Between classes and during the after hours cycle, the system is configured to quickly exhaust the interior space to replace the interior air with natural, fresh air. This may be achieved manually or automatically or a combination of both by opening doors or windows to an exterior environment and activating a fan or air flow system of vents.

A plurality of sensors positioned in a variety of locations throughout the interior space constantly or periodically measure a plurality of parameters, including, but not limited to temperature, humidity, particulate count, and air flow. The plurality of sensors are coupled to a central processing unit through wired or wireless communication techniques.

The data gathered by the plurality of sensors is compared, within the central processing unit to threshold levels for each of the detected parameters. If one or more of the sensors detects a level that is above or below the respective threshold, the processing unit will alert a system manager or may automatically implement an adjustment protocol.

Carbon dioxide level and particulates in the air may be the most aggressively monitor parameters. A sensor may be included within or adjacent to the doorway to count a number of participants currently within the space. The number of participants in the space and the type of class can provide the system with information to determine an appropriate frequency for detecting carbon dioxide levels and proactively vent or bring in fresh air as levels begin increasing.

Other components within the system include an energy recovery ventilator and an ionic filtration system. The ionic filtration system may be a bipolar ionization system that are within the environment treatment unit. A filter, such as a MERV filter, for example, MERV-13 or higher, may be positioned downstream from the ionization system.

### BRIEF DESCRIPTION OF THE FIGURES

In the figures, the sizes and relative positions of elements in the figures are not necessarily drawn to scale. For example, the shapes of various elements and angles are not drawn to scale, and some of these elements are enlarged and positioned to improve figure legibility.

FIG. 1 shows a perspective view of an embodiment of an environment management system.

FIG. 2 shows a block diagram of an embodiment of an environment management system.

FIG. 3 shows a top down view of an embodiment of an environment management system.

### DETAILED DESCRIPTION

Unless the context requires otherwise, throughout the specification and claims which follow, the word “comprise” and variations thereof, such as “comprises” and “comprising” are to be construed in an open, inclusive sense, that is, as “including but not limited to”.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the context clearly dictates otherwise. It should also be noted that the term “or” is generally employed in its broadest sense, that is, as meaning “and/or” unless the context clearly dictates otherwise.

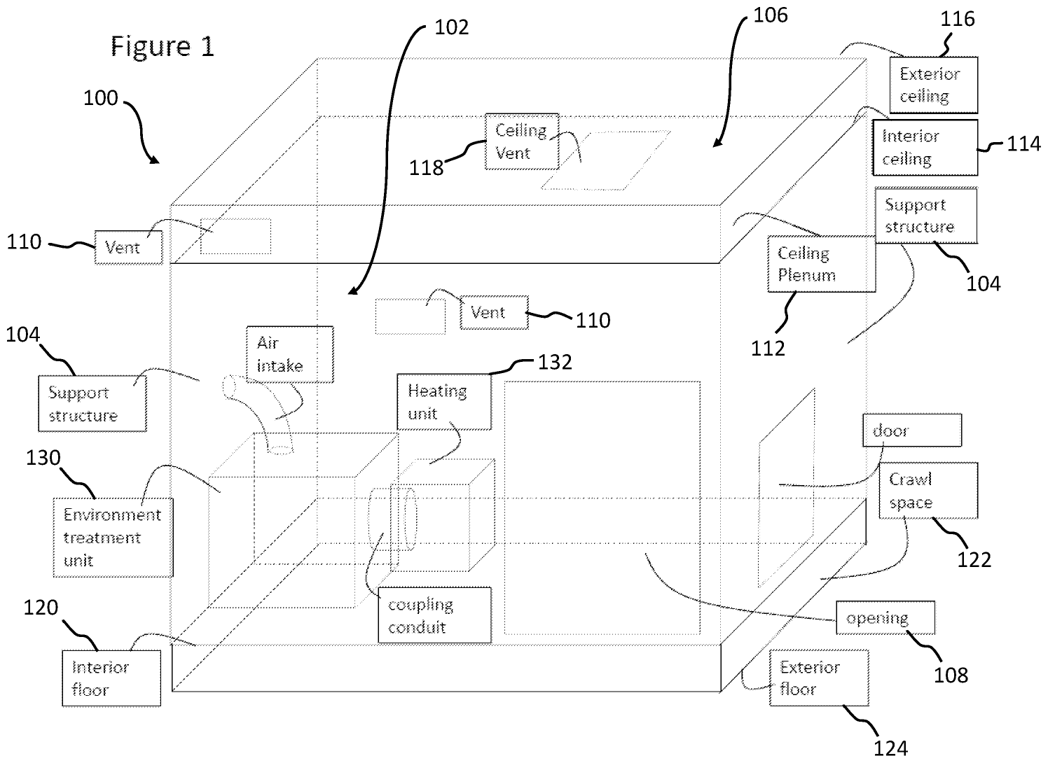

Provided according to one or more embodiments are systems, methods, and networks to control an environment management system 100. The environment management system 100 according to one embodiment is depicted in FIG. 1. The environment management system 100 is configured to sense and modulate various environmental factors or air quality parameters in an interior area 102, such as a yoga studio or other interior exercise space. When a mammal's body, is exercising and undergoing aerobic respiration, the lungs are taking in a greater volume of air to supply the body with an increased amount of oxygen to meet the increased demand from the tissues in the body, such as muscle tissue. The more intense the exercise the greater the aerobic needs of the body, and the more air is inhaled to supply the increased demand for oxygen. The increased volume of air inhaled during exercise can be more than five times the volume of air breathed when sedentary. Removing contaminants from the air, such as volatile organic compounds, or carbon monoxide, may lead to improved performance and prevent damage to the lungs during increased levels of exertion. As an added hindrance to maintaining air quality in indoor spaces, such as an indoor gym or yoga studio, multiple people will be in and out of the space, all breathing deeply throughout the day. This increased traffic will increase the levels of contaminants such as excess carbon dioxide or particulate matter in the indoor space.

A non-limiting list of air quality parameters monitored and controlled by the environment management system 100 includes air flow, temperature, humidity, carbon dioxide, nitrogen dioxide, air pressure, ozone, carbon monoxide, particulate matter, and total concentration volatile organic compounds (TVOC). The environment management system 100 is also configured to diminish or eliminate air contaminants in the interior area 102, which could be an area that includes a regularly changing group of humans through out a day and could include an intentional fluctuating temperature that can generate moisture in the area, such as a heated yoga environment or a spin or stationary bike studio.

The air in the interior area 102 will be understood by those of ordinary skill in the art to include a mixture of gases including but not limited to nitrogen, oxygen, water vapor, carbon dioxide, argon, and other minor components. The air in the interior area 102 is sufficient for mammals to breath safely. In some embodiments the air in the interior area 102 is optimized to be breathed by mammals, including humans. The optimization of the air in the interior area 102 may include, but is not limited to maintaining predetermined levels of particulate matter, temperature, ozone, carbon monoxide, water vapor (humidity), carbon dioxide, nitrogen dioxide, or oxygen. Among the potential contaminants the environment management system will diminish or eliminate include TVOC, ozone, carbon monoxide, particulate matter, or potential pathogenic organisms including, but not limited to, bacteria, viruses, or fungi.

The interior area 102 in which the environment management system 100 operates is made up of a plurality of lateral support structures 104 each of which has a first side and a second side opposite the first side. In some embodiments these lateral support structures 104 are walls. The lateral support structure 104 has a top and bottom opposite the top. The top of the lateral support structure 104 is coupled to a ceiling 106. In some embodiments the lateral support structure 104 has at least one opening 108, i.e, a window or a door or other opening to an exterior environment from the interior area. In some embodiments the lateral support structure 104 will have more than one opening 108. In some embodiments the window or door will span from the top to the bottom of the lateral support structure 104. The window or door will open the internal area 102 to allow for an exchange of air from outside the internal area 102. The internal area 102 may be air tight and may not exchange any gases external to the internal area 102. Alternatively, the internal area 102 will be air tight, but allow for the passage of gases through a plurality of vents 110 that are manually or automatically opened in response to a threshold condition being met. In some embodiments, the air pressure in the internal area 102 is maintained higher than the air pressure outside the internal area 102 so any gases passing through the plurality of vents 110 will move out of the internal area 102. The vents 110 can change from an open configuration to a closed configuration. In some embodiments the vents 110 will change from an open configuration to a closed configuration with a gear system and a servo or other controller.

As can be seen in FIG. 1, the environment management system may also include a ceiling plenum area 112 between an internal ceiling 114 and an external ceiling 116. The internal ceiling 114 has a first surface and a second surface opposite the first surface. The internal ceiling 114 first surface faces the internal area 102 and the internal ceiling 112 second surface faces the ceiling plenum area 112. The external ceiling 116 also has a first side and a second side opposite the first side. The external ceiling 116 first side faces the ceiling plenum area 112. In some embodiments the ceiling plenum area 112 is in fluid communication with the internal area 102 through a ceiling vent 118. The air pressure in the ceiling plenum area 112 may be lower than the air pressure in the internal area 102 which will lead to the flow of gases from the internal area 102 to the ceiling plenum 112. In some embodiments the interior ceiling 114 provides an air tight barrier between the interior area 112 and the ceiling plenum area 114. In some embodiments the interior ceiling 114 is tiled with material that is easy to clean and resists attachments of contaminants such as fungi or bacteria.

The environment management system 100 may also include an interior floor 120 coupled to bottom of the lateral support structure 104. The interior floor 120 has a first side and a second side opposite the first side. The interior floor 120 may be waterproof and seamless to prevent intrusion of dirt, moisture, or contaminants such as fungi, bacteria, or viruses. The interior floor 120 may be composed of various layers. One of the layers of the interior floor 120 may be cork to provide a soft feel while acting as a retardant to contaminants such as fungi, bacteria, or viruses. The environment management system 100 may also include a crawl space 122, such that the first side of the interior floor 120 is facing the interior area 102 and the second side is facing the crawl space 122. An exterior floor 124 that has a first side and a second side where the first side of the exterior floor 124 is facing the crawl space 122. In some embodiments, there may be no vents connecting the interior area 102 and the crawl space 122 so there is no exchange of gases between the interior area 102 and the crawl space 122. Alternatively, there may be an opening in the interior floor 120 configured to permit access to the crawl space 122. When the opening in the interior floor 120 is closed there is no exchange of gases between the interior area 102 and the crawl space 122.

The environment management system 100 may also include at least one contaminant resistant material. The contaminant resistant material may resist attachment of contaminants such as bacteria, viruses, fungi, inorganic chemicals, or organic chemicals. In some embodiments the contaminant resistant material will alter the porosity of the surface it covers. The components in the environment management system 100 may be composed of contaminant resistant materials. The first surface and second surface of the interior ceiling 114 may be covered by a first contaminant resistant material. The first surface of the exterior ceiling 116 may be covered by a second contaminant resistant material. The first side of the interior ceiling 114 may be covered by a first contaminant resistant material and the second side of the interior ceiling 114 may be covered by a second contaminant resistant material. The interior ceiling 114 may also be made from a contaminant resistant material. The contaminant resistant material may contain antimicrobial agents. Alternatively, the interior floor 120 may be composed of contaminant resistant material. The interior floor 120 may be covered with contaminant resistant material.

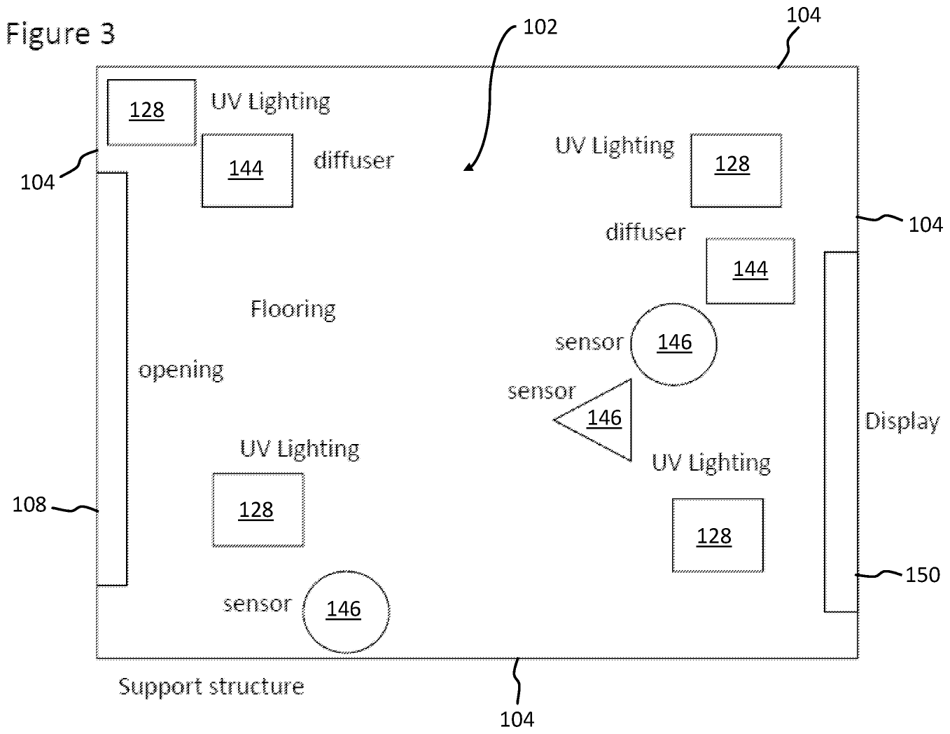

In some configurations, the environment management system 100 may include an ultraviolet (UV) light disinfecting system, which may include ultraviolet lights 128 as shown in FIG. 3. The wavelength of UV radiation ranges from 328 nm to 210 nm (3280 A to 2100 A). Its maximum bactericidal effect occurs at 240-280 nm. The germicidal effectiveness and use of UV radiation is influenced by organic matter, wavelength, temperature, type of microorganism, and UV intensity. In some embodiments ones of the ultraviolet lights 128 in the interior area 102 may be configured with ultraviolet-C (UVC) lamps. Alternatively the ceiling plenum 112 and the crawl space 122 may also be configured with UVC lamps. The ultraviolet lights 128 in the interior area 102 may also be configured with ultraviolet-A (UVA) or ultraviolet-B (UVB) lamps. The ceiling plenum and the crawl space 122 may be configured with UVA or UVB lamps. The ceiling plenum 112, the interior area 102, and the crawl space 122 may all be configured with UVA lamps, UVB lamps, UVC lamps, or any combination of these lamps. The UVA, UVB, or UVC lamps may be coupled to the lateral structural supports 104, the interior ceiling 114, or the interior floor 120.

The environment management system 100 includes an environment treatment unit 130 which may be composed of blowers, motors, fans, ionic filtration elements, and particle filtration elements. Filtration elements are rated by their ability to collect particulate size particulates. Filters may be rated according to a Minimum Efficiency Reporting Value (MERV) which indicated the efficiency of a filter to capture particles of varying sizes. The higher the MERV rating, the higher the air filtration capabilities of a particular filter. Filters are configured with spaces between filter fibers, which will capture only certain size particulates and allow others to pass through the filter. One of ordinary skill in the art will appreciate that High Efficiency Particulate Air (HEPA) grade filters are rated from 14 to 17 MERV. A 99.97% HEPA filter will remove all particulates grater than 0.3 microns. A lesser grade HEPA such as the 95% will remove particulates grater than 5.0 microns. The higher the MERV rating the greater the amount of air pressure it takes to pass gases through the filter. The higher MERV rating will reduce air flow through the filter and reduce air velocity of the gases exiting the filter.

The environment treatment unit 130 may also be composed of an ionic filtration element. The ionic filtration element may filter out particles as small as 0.01 microns in size. These particles may include pollen, bacteria, allergens, and dust. In some embodiments the ionic filtration element will emit negatively charged ions into the air. These negatively charged ions may attract positively charged particulates, such as the pollen, bacteria, allergens, and dust. Once this bond is formed the particles becomes heavier than they were before the bond, and may become trapped on an electrostatic collection plate.

Both the ionic filtration element and the particle filtration element can be removed from the environment treatment unit 130 to facilitate maintenance, such as cleaning. The environment treatment unit 130 may have the ionic filtration element and particle filtration elements configured in series or in parallel. The ionic filtration element may be placed first so the air flow moves through the ionic filtration element first before passing through the particle filtration element. The particle filtration element may be placed first so the air flow moves through the particle filtration element first before passing through the ionic filtration element.

In another embodiment the environment management system 100 may include a heating unit 132. The heating unit 132 may be a convection heater, where electricity passes through a heating element causing the heating element to become hot. The heating element may be made of metal or ceramic. The process of heating a heating element with electricity may be known as joule heating. The heat from the joule heating is then transferred to the air in the interior area by convection. The heating unit 132 may use infrared heating by passing electricity through a conductive wire to heat it, the heat is then given off as radiant heating. The heated wire emits infrared rays. The heating unit 132 may be coupled to a solar thermal collector. The solar thermal collector heats fluid by absorbing sunlight and this heated fluid then passes into the heating unit 132 which then emits radiant heat. The heating unit 132 may capture heat and humidity from exhaust air 133 generated in the interior area 102. The heating unit 132 may use any combination of these heating methods.

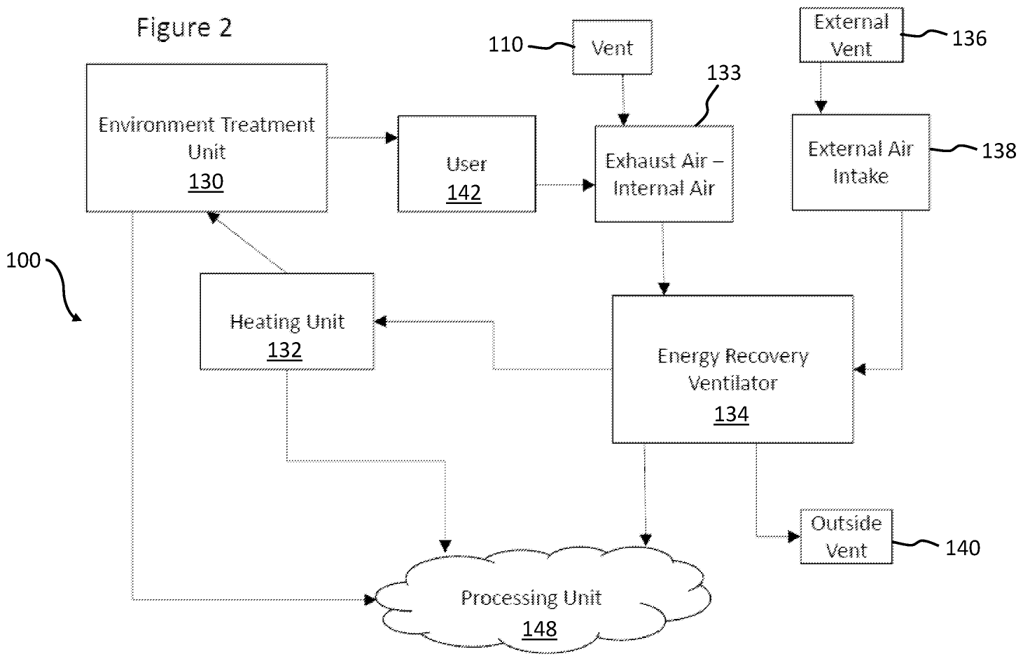

In another embodiment the environment management system 100 includes an Energy Recovery Ventilator (ERV) 134. The ERV 134 receives air flow from an external vent 136 through an external air intake 138, as depicted in FIG. 2. The ERV 134 may also receive air from the interior area 102 that has mixed with air entering the interior area 102 through another external vent. The ERV 130 may then recover heat and humidity from the air and transfer this heat and humidity to the heating unit 132. The ERV 134 may also shunt exhaust air 133 out of the interior area 102 through an outside vent 140. The heat and humidity transferred to the heating unit 132 can be used to heat or modify the humidity of the air flowing through the heating unit 132. The heating unit 132 may process air that is transferred to the environment treatment unit 130 to then be released into the interior area 102 to be breathed by users 142 in the interior area 102. The users 142 in the interior area 102 then exhale exhaust air 133 that is either vented out of the interior area 102 through an outside vent 140 or is taken up by the ERV 134.

The heating unit 132 may be coupled to the environment treatment unit 130. Air may flow through the environment treatment unit 130 before passing through the heating unit 132. Air may flow through the heating unit 132 before passing through the environment treatment unit 130. The environment management system 100 may include diffusers 144, as depicted in FIG. 3, so individuals in the interior area 102 can use aroma therapy.

The blowers, motors and fans in the environment treatment unit 130 may be configured to maintain air flow through the environment treatment unit 130 and into the interior area 102. The environment treatment unit 130 may be in fluid communication with the exterior of the internal area 102 through the external air intake 138. The environment treatment unit 130 may be in fluid communication with the interior area 102. The environment treatment unit 130 may receive gases from either the interior area 102 or external to the interior area 102 or a combination of both. The environment treatment unit 130 may release treated gases into the interior area 102. The treated gas may have a predetermined levels of, for example, humidity, temperature, carbon dioxide, nitrogen dioxide, air pressure, ozone, carbon monoxide, particulate matter, or TVOC. The efficiency of the environment treatment unit 130 can be tracked by the amount of Air Changes per Hour (ACPH or ACH) which is a measure of the air volume added or removed from a space, such as the interior area 102.

The environment management system 100 may include sensors 146, as shown in FIG. 3, for an air flow, temperature, humidity, carbon dioxide, nitrogen dioxide, air pressure, ozone, carbon monoxide, particulate matter, and total concentration volatile organic compounds (TVOC). These sensors 146 may be directly coupled to the environment treatment unit 130 or may be connected through a network connection to the environment treatment unit 130. The sensors 146 may be grouped together or may be spread apart from each other. There may be more than one sensor for each of the types of sensors 146, as a non-limiting example, there may be four temperature sensors placed throughout the interior area 102, including on the lateral support structures 104, by the opening in the lateral support structure 104, on the interior ceiling 114, or the interior floor 120. Any combination of sensors 146 placed throughout the interior area 102 is contemplated. There may also be sensors 146 placed in the ceiling plenum 112 or in the crawl space 122 or in both. Various combinations of sensors 146 may be placed in the interior area 102, the ceiling plenum 112, and the crawl space 122.

The environment management system 100 may include a processing unit 148 configured to receive data input from at least one sensor 146. The data generated by the sensors 146 in the interior area 102, the ceiling plenum 112, or the crawl space 122 may be transmitted to the processing unit 148, such as through a network. The processing unit 148 can then translate this data into visual data to be displayed on a display in the interior area 102. Alternatively, there are multiple displays 150 throughout the interior area 102. The display 150 may be large enough to show the data sent by the processing unit 148 to any user 142 within the interior area 102. The display 150 may be attached to a lateral support structure 104. The display 150 may be a digital display. For example, the display 150 may display current temperature, TVOC, nitrous oxide levels, carbon dioxide levels, and a virus index, which may reference a likelihood of viruses being present based on other conditions of the air.

The environment management system 100 may include a user interface (not shown) coupled to the processing unit 148. The environment management system 100 can operate in response to manual settings input by a user 142. The processing unit 148 can make real-time adjustments to other components of the environment management system 100 such as the environment treatment unit 130 or the heating unit 132. As a non-limiting example, the processing unit 148 may receive data from one or more of the sensors 146, which may be temperature sensors to determine whether the temperature has increased past a set threshold value. The processing unit 148 may then use this data to make real-time adjustments to the heating unit 132 to turn off the heating unit 132 and real-time adjustments to the environment treatment unit 130 to increase the air flow.

The components of the environment management system 100 cooperate together to provide an environment to individuals in which various parameters are monitored and controlled. The individuals using the environment management system 100 may include attendees to an exercise class, such as a yoga class. Alternatively, the individuals using the environment management system 100 may be a healthcare provider in an environment such as a surgical suite.

The processing unit 148 can include processing resources, memory resources, data transmission resources, displays, input devices, or other systems and devices that may be used to monitor and control various parameters in an environment management system 100.

In one embodiment, the processing unit 148 can store parameters related to the air quality in the interior area 102. The parameters related to air quality can be temperature, humidity, air pressure, carbon dioxide, TVOC, particulate matter, carbon monoxide, nitrogen dioxide, an ozone. This information may be stored in one or more databases in computer memories at least partially located in the environment management system 100.

In one embodiment, an individual may interact with a user interface, such as an interface on the environment treatment unit 130. The individual may be monitoring various parameters on the display 150 which indicates the real-time levels of, for example, the temperature, humidity, air pressure, carbon dioxide, TVOC, particulate matter, carbon monoxide, nitrogen dioxide, or ozone. The individual may make adjustments to the environment treatment unit 130 or the heating unit 132 based on the data presented on the display 150.

In one embodiment, the processing unit 148 may record details regarding the air quality and the response the user is making based on the air quality parameters. As a non-limiting example, the processing unit 148 may record that the temperature setting on the heating unit 132 is reduced when the temperature in the interior area 102 rises above a specific threshold value which is sensed by one or more of the sensors 146 and displayed on the display 150. The processing unit 148 may use these recorded details to automatically implement changes to the environment treatment unit and the heating unit 132 when threshold values of the air quality parameters are reached in the interior area 102.

Alternatively, after receiving the data from one or more of the sensors 146, the processing unit 148 may automatically make an adjustment to any one of the environment treatment unit 130, the heating unit 132, the vents 110, or the opening 108 in the lateral support structure.

In one embodiment, some of the information associated air quality and the parameters monitored by the sensors 146 in the environment management system 100 may be recorded by the processing unit 148. The processing unit 148 can include computing resources including, but not limited to servers, processing resources, memory resources, and data transmission resources. The processing unit 148 can include physical and virtual computing resources.

In one embodiment, the processing unit 148 can be, at least partially, a cloud-based system. The processing unit 148 can include medical information databases, air quality applications, and other information resources implemented with the computing resources. These databases, applications, and resources may be implemented partially or entirely in the cloud. These databases, applications, and resources may be accessible via the cloud.

In one embodiment, the environment management system 100 and the processing unit 148 may share software applications with computer systems remote to the environment management system 100. The shared software applications may enable data stored at location of the environment management system 100 to be automatically sent and recorded in to a remote computing system (not shown). Likewise, the shared software applications may enable the processing unit in the environment management system 100 to retrieve information related to air quality or components in the environment management system 100 from remote computing systems.

In one embodiment, an individual that will use the environment management system 100 will enter the interior area 102 through the opening 108 in the lateral support structure 104. The ultraviolet (UV) lights 128 will be configured to turn off when an individual enters the interior area 102. The individual can then engage the user interface either direct or remotely coupled to the environment treatment unit 130 to turn on the environment treatment unit 130 or adjust any of the air quality parameters. The individual may also view the levels of various air quality parameters on the display 150. The display 150 may be coupled to the lateral support structure 104. In some embodiments the processing unit 148 in the environment management system 100 may be in communication with a handheld device, such as an individual's personal phone, and display the air quality parameters on an application on the phone. In an alternative embodiment the individual may engage the environment management system 100 to verify that the processing unit 148 is automatically adjusting the air quality parameters according to threshold levels of the air quality parameters. As a non-limiting example, the individual may engage the user interface to verify that the temperature displayed on the display has been maintained automatically by the heating unit 132 in response to changes in temperature monitored by one or more of the sensors 146, at least one of which being a temperature sensor, in the interior area 102.

In one embodiment, the environment treatment unit 130 includes computing resources such as processing resources, memory resources, data transmission resources, or other types of computing resources. These computing resources can receive, process, and store data related to air quality, such as temperature and carbon dioxide, that have been provided by the sensors 146 coupled to the processing unit 148. The computing resources of the environment treatment unit 130 may include databases that store these types of information. Various encryption and data security methods will be implemented to ensure safe storage of any data collected by the processing unit 148. The data may not be permanently stored and instead will only be accessed and temporarily saved at the environment treatment unit 130 when needed by the individual.

In one embodiment, the computing resources of the environment management system 130 may assist in obtaining and analyzing current air quality parameters in the interior area 102. These indicators may include temperature, air flow, humidity, carbon dioxide, nitrogen dioxide, air pressure, ozone, carbon monoxide, particulate matter, or TVOC. In one embodiment, the computing resources of the environment management system 100 may include one or more machine learning-based analysis models. The machine learning-based analysis models may be trained, with one or more machine learning processes, to determine whether an air quality parameter, such as temperature, should be adjusted. The machine learning processes may train the analysis model to make these determinations based on the current physical parameters monitored by the one or more sensors 146, at least some of which are coupled to the processing unit 148.

In one example, when a user accesses the user interface to attempt to adjust one of the air quality parameters, sensors of the environment management system 100 may sense current physical indicators in the interior area 102. These current physical indicators may pass to the analysis model. The analysis model may determine that based on the current physical state of the interior area 102, the air quality parameter should not be adjusted. Instead, the analysis model may determine that no parameters should be changed or that a different air quality parameter should be adjusted. For example, if a user attempts to adjust the temperature the analysis model may determine that the temperature should remain the same but humidity should instead be adjusted to achieve a set of threshold levels either preprogrammed into the processing unit or that the analysis model has learned will result in the desired air quality parameters.

In one embodiment, the processing unit 148 enables communication between the remote computing systems, the computing resources of the environment treatment unit, and the environment management system. The networks can include the combination of one or more of local area networks, wireless area networks, cellular networks, satellite communication networks, the Internet, or other types of communication networks.

An embodiment of an environment management system of the present disclosure may be summarized as including: an interior area surrounded by a plurality of lateral support structures, each of the plurality of support structures has a first surface and a second surface, and a top and a bottom; an opening in at least one of the plurality of support structures that provides access from the interior area to an exterior area; an exterior ceiling; a ceiling plenum area; an interior ceiling coupled to the top of the plurality of support structures, the interior ceiling has an interior ceiling first surface and an interior ceiling second surface opposite the interior ceiling first surface, the ceiling plenum area is between the interior ceiling and the exterior ceiling; a first contaminant resistant material is on the interior ceiling first surface; a second contaminant resistant material is on the exterior ceiling within the ceiling plenum area; a ceiling vent coupled between the ceiling plenum area and the interior area; an interior floor coupled to the bottom of the plurality of support structures; a plurality of exterior vents coupled between the interior area and the exterior area; an ultraviolet light treatment system in the interior area; an environment treatment unit in the interior area in fluid communication with the exterior area, the environment treatment unit including: an ionic filtration element; and a particle filtration element; a heating unit in fluid communication with the environment treatment unit; a temperature sensor coupled to the environment treatment unit; an air flow sensor coupled to the environment treatment unit; a humidity sensor coupled to the environment treatment unit; a carbon dioxide sensor coupled to the environment treatment unit; a nitrogen dioxide sensor coupled to the environment treatment unit; an air pressure sensor coupled to the environment treatment unit; an ozone sensor coupled to the environment treatment unit; a carbon monoxide sensor coupled to the environment treatment unit; a particulate matter sensor coupled to the environment treatment unit; a total concentration of volatile organic compounds (TVOC) sensor coupled to the environment treatment unit; a processing unit coupled to the temperature sensor, air flow sensor, humidity sensor, carbon dioxide sensor, nitrogen dioxide sensor, air pressure sensor, ozone sensor, carbon monoxide sensor, particulate matter sensor, TVOC sensor, the environment treatment unit, the ultraviolet light treatment system, and the heating unit, the processing unit being configured to control the environment treatment unit and the heating unit; a user interface coupled to the processing unit; wherein the processing unit controls the environment treatment unit and the heating unit based on the readings provided by the temperature sensor, air flow sensor, humidity sensor, carbon dioxide sensor, nitrogen dioxide sensor, air pressure sensor, ozone sensor, carbon monoxide sensor, particulate matter sensor, TVOC sensor.

The environment management system may further include a plurality of displays coupled to the processing unit within the interior area to display visual data transmitted from the processing unit in real-time.

The environment management system may further include an air intake system coupled to the environment treatment unit to pump air from outside the interior area into the environment treatment unit, where the air intake system is configured to pump gas against an air pressure gradient.

The environment management system may further include ultraviolet (UV) lights in the ceiling plenum, where the UV lights in the ceiling plenum may be UVA, UVB, or UVC lamps.

At least one of the openings in the lateral support structure may be a door.

The interior ceiling first surface may be facing the interior area and the interior ceiling second surface may be facing a ceiling plenum area.

The exterior ceiling may have an exterior ceiling first surface facing the ceiling plenum area and may have an exterior ceiling second surface opposite the exterior ceiling first surface.

The ceiling vent may be configured to be adjustable between a closed position and an open position in response to input from the processing unit to prevent fluid communication between the ceiling plenum area and the interior area during specific conditions, such as when the temperature reaches a threshold value in the interior area.

The plurality of exterior vents may be configured to be adjustable between a closed position and an open position in response to input from the processing unit to prevent fluid communication from the interior area to exterior to the interior area during specific conditions, such as when the temperature reaches a threshold value in the interior area.

The environment management system may further include a crawl space, wherein the interior floor has an interior floor first side and an interior floor second side opposite the interior floor first side, wherein the interior floor first side is facing the interior area, and wherein the interior floor second side is facing a crawl space.

The environment management system may further include an exterior floor, wherein the exterior floor has an exterior floor first side and an exterior floor second side opposite the interior floor first side, the exterior floor first side is facing the crawl space and the exterior floor second side is facing the exterior areas.

The processing unit may be configured to automatically adjust the environment treatment unit, the ultraviolet light treatment system, or the heating unit in response to at least one of the sensors for air flow, temperature, humidity, carbon dioxide; nitrogen dioxide, air pressure, ozone, carbon monoxide, particulate matter, or total concentration volatile organic compounds (TVOC) reaching predetermined threshold levels.

The sensors may be for articulate options for where the sensors air flow, temperature, humidity, carbon dioxide; nitrogen dioxide, air pressure, ozone, carbon monoxide, particulate matter, or total concentration volatile organic compounds (TVOC) are coupled either directly or indirectly through a network connection to the processing unit.

The environment management system further includes an energy recovery ventilator that recovers heat from exhaust gases produced in the interior area and transfers this heat to the heating unit to heat gases passing through the heating unit.

An embodiment of an environment management system of the present disclosure may be summarized as including: an interior area; an ultraviolet light treatment system in the interior area; an environment treatment unit in the interior area, including: an ionic filtration element; a particle filtration element; a heating unit in fluid communication with the environment treatment unit; a processing unit coupled to the ultraviolet light treatment system, the environment treatment unit, and the heating unit; a sensor configured to monitor an air quality parameter coupled to the processing unit; and a display coupled to the processing unit which displays the air quality parameters monitored by the sensor in real-time.

The environment management system may further include an air intake system coupled to the environment treatment unit to pump air from outside the interior area into the environment treatment unit, where the air intake system is configured to pump gas against an air pressure gradient.

The environment management system may further include a plurality of exterior vents configured to move from a closed position to an open position to permit fluid communication between the interior area and the area outside the interior area.

The sensor may be at least one of a temperature sensor, an air flow sensor, a humidity sensor, a carbon dioxide sensor, a nitrogen dioxide sensor, an air pressure sensor, an ozone sensor, a carbon monoxide sensor, a particulate matter sensor, and a total concentration of volatile organic compounds sensor.

The environment management system may further include a user interface coupled to the processing unit.

1. An environment management system, comprising:
an interior area surrounded by a plurality of lateral support structures, each of the plurality of support structures has a first surface and a second surface, and a top and a bottom;
an opening in at least one of the plurality of support structures that provides access from the interior area to an exterior area;
an exterior ceiling;
a ceiling plenum area;
an interior ceiling coupled to the top of the plurality of support structures, the interior ceiling has an interior ceiling first surface and an interior ceiling second surface opposite the interior ceiling first surface, the ceiling plenum area is between the interior ceiling and the exterior ceiling;
a first contaminant resistant material is on the interior ceiling first surface;
a second contaminant resistant material is on the exterior ceiling within the ceiling plenum area;
a ceiling vent coupled between the ceiling plenum area and the interior area;
an interior floor coupled to the bottom of the plurality of support structures;
a plurality of exterior vents coupled between the interior area and the exterior area;
an ultraviolet light treatment system in the interior area;
an environment treatment unit in the interior area in fluid communication with the exterior area, the environment treatment unit including:
an ionic filtration element;
a particle filtration element;
a heating unit in fluid communication with the environment treatment unit;
a temperature sensor coupled to the environment treatment unit;
an air flow sensor coupled to the environment treatment unit;
a humidity sensor coupled to the environment treatment unit;
a carbon dioxide sensor coupled to the environment treatment unit;
a nitrogen dioxide sensor coupled to the environment treatment unit;
an air pressure sensor coupled to the environment treatment unit;
an ozone sensor coupled to the environment treatment unit;
a carbon monoxide sensor coupled to the environment treatment unit;
a particulate matter sensor coupled to the environment treatment unit;
a total concentration of volatile organic compounds (TVOC) sensor coupled to the environment treatment unit;
a processing unit coupled to the temperature sensor, air flow sensor, humidity sensor, carbon dioxide sensor, nitrogen dioxide sensor, air pressure sensor, ozone sensor, carbon monoxide sensor, particulate matter sensor, TVOC sensor, the environment treatment unit, the ultraviolet light treatment system, and the heating unit, the processing unit being configured to control the environment treatment unit and the heating unit;
a user interface coupled to the processing unit;
wherein the processing unit controls the environment treatment unit and the heating unit based on the readings provided by the temperature sensor, air flow sensor, humidity sensor, carbon dioxide sensor, nitrogen dioxide sensor, air pressure sensor, ozone sensor, carbon monoxide sensor, particulate matter sensor, TVOC sensor.

2. The environment management system of claim 1, further comprising a plurality of displays coupled to the processing unit within the interior area to display visual data transmitted from the processing unit in real-time.

3. The environment management system of claim 1, further comprising an air intake system coupled to the environment treatment unit to pump air from outside the interior area into the environment treatment unit, where the air intake system is configured to pump gas against an air pressure gradient.

4. The environment management system of claim 1, further comprising ultraviolet (UV) lights in the ceiling plenum, where the UV lights in the ceiling plenum may be UVA, UVB, or UVC lamps.

5. The environment management system of claim 1, wherein at least one of the openings in the lateral support structure is a door.

6. The environment management system of claim 1, wherein the interior ceiling first surface is facing the interior area and the interior ceiling second surface is facing a ceiling plenum area.

7. The environment management system of claim 1, wherein the exterior ceiling has an exterior ceiling first surface facing the ceiling plenum area and an exterior ceiling second surface opposite the exterior ceiling first surface.

8. The environment management system of claim 1, wherein the ceiling vent is configured to be adjustable between a closed position and an open position in response to input from the processing unit to prevent fluid communication between the ceiling plenum area and the interior area during specific conditions, such as when the temperature reaches a threshold value in the interior area.

9. The environment management system of claim 1, wherein the plurality of exterior vents is configured to be adjustable between a closed position and an open position in response to input from the processing unit to prevent fluid communication from the interior area to exterior to the interior area during specific conditions, such as when the temperature reaches a threshold value in the interior area.

10. The environment management system of claim 1, further comprising a crawl space, wherein the interior floor has an interior floor first side and an interior floor second side opposite the interior floor first side, wherein the interior floor first side is facing the interior area, and wherein the interior floor second side is facing a crawl space.

11. The environment management system of claim 1, further comprising an exterior floor, where the exterior floor has an exterior floor first side and an exterior floor second side opposite the interior floor first side, where the exterior floor first side is facing the crawl space and the exterior floor second side is facing the exterior areas.

12. The environment management system of claim 1, wherein the processing unit is configured to automatically adjust the environment treatment unit, the ultraviolet light treatment system, or the heating unit in response to at least one of the sensors for air flow, temperature, humidity, carbon dioxide; nitrogen dioxide, air pressure, ozone, carbon monoxide, particulate matter, or total concentration volatile organic compounds (TVOC) reaching predetermined threshold levels.

13. The environment management system of claim 1, wherein the sensors for articulate options for where the sensors air flow, temperature, humidity, carbon dioxide; nitrogen dioxide, air pressure, ozone, carbon monoxide, particulate matter, or total concentration volatile organic compounds (TVOC) are coupled either directly or indirectly through a network connection to the processing unit.

14. The environment management system of claim 1, further comprising an energy recovery ventilator that recovers heat from exhaust gases produced in the interior area and transfers this heat to the heating unit to heat gases passing through the heating unit.

15. An environment management system, comprising:
an interior area;
an ultraviolet light treatment system in the interior area;
an environment treatment unit in the interior area, including:
an ionic filtration element;
a particle filtration element;
a heating unit in fluid communication with the environment treatment unit;
a processing unit coupled to the ultraviolet light treatment system, the environment treatment unit, and the heating unit;
a sensor configured to monitor an air quality parameter coupled to the processing unit; and
a display coupled to the processing unit which displays the air quality parameters monitored by the sensor in real-time.

16. The environment management system of claim 15, further comprising an air intake system coupled to the environment treatment unit to pump air from outside the interior area into the environment treatment unit, where the air intake system is configured to pump gas against an air pressure gradient.

17. The environment management system of claim 15, further comprising a plurality of exterior vents configured to move from a closed position to an open position to permit fluid communication between the interior area and the area outside the interior area.

18. The environment management system of claim 15, wherein the sensor is at least one of a temperature sensor, an air flow sensor, a humidity sensor, a carbon dioxide sensor, a nitrogen dioxide sensor, an air pressure sensor, an ozone sensor, a carbon monoxide sensor, a particulate matter sensor, and a total concentration of volatile organic compounds sensor.

19. The environment management system of claim 15, further comprising a user interface coupled to the processing unit.
